(19) United States
(12) Patent Application Publication
(10) Pub. No.: US 2015/0338388 A1
(43) Pub. Date: Nov. 26, 2015

(54) SAFETY KIT FOR TREATING BIOLOGICAL MATERIAL

(71) Applicant: Mauro PEPE, Roma (IT)

(72) Inventor: Mauro PEPE, Roma (IT)

(21) Appl. No.: 14/646,681

(22) PCT Filed: Nov. 26, 2013
(86) PCT No.: PCT/IT2013/000330

§ 371 Date: May 21, 2015

(30) Foreign Application Priority Data
Nov. 26, 2012 (IT) RM2012A000592

(51) Int. Cl. G01N 33/49; B65B 3/12; B65B 31/00; B65B 3/00

(52) U.S. Cl. CPC G01N 33/49; B65B 3/003; B65B 3/12; B65B 31/00

(57) ABSTRACT

Safety kit for treating biological material for treating venous blood, adapted to facilitate the transfer of blood from a common syringe to a common collection test tube, the latter having at its interior a predefined vacuum or inert atmosphere, including at least one tubular protection casing, preferably cylindrical, at least one component assemblable or already assembled inside the protection casing. The protection casing and the component, when assembled, adapted to constitute the connection element between the syringe and the collection test tube, and at least one package adapted to contain the components of the kit, wherein the protection casing has at least two portions both portions with equivalent extension and adapted to receive the component, the component having at least two projections the projection extended through the portion and the projection extended into the portion and respectively adapted to connect with the syringe and with the collection test tube.

### FIELD OF THE ART

The present invention relates to a new kit of components for drawing biological material and preferably for drawing blood. More in detail, the present invention relates to a kit comprising components adapted to facilitate, under safe conditions, the transfer of blood from a common syringe to a common collection test tube. The kit, object of the invention, also provides for the use both of primary test tubes, having a predetermined vacuum therein, and test tubes, less costly, having an inert atmosphere therein.

### STATE OF THE ART

Up to now, considerations on the devices used for drawing biological material, and in particular for drawing venous blood, mainly regard regulations relative to patient and operator safety and evaluations of technical and economical nature.

In particular, it is advised to use devices that provide for the integration of 20-gauge or 21-gauge needles, systems such as “holders, adaptors or “jackets” and of primary test tubes preferably having a predetermined vacuum therein.

In some cases, a possible alternative is represented by syringes, for example in emergency situations in which it is not possible to find the above devices on the market, or in particular anatomic and/or physical situations that make it impossible or inadvisable to use such devices. For example, it is preferable to use syringes in the case in which the drawing of venous blood must be made on a patient who has veins that are easily collapsible when subjected to vacuum, and more in detail when subjected to the low pressure present in the primary tube. In these cases, it is then necessary to graduate the suction, therefore the use of a syringe is preferable with respect to the use of integrated systems of needles such as butterfly needles. When the drawing is executed with a common syringe, it is also advisable to draw not more than 20 ml of blood. Once drawn, the blood must subsequently be transferred into the test tube in a manner such to make the blood flow along the wall of the test tube in order to prevent the risk of hemolysis. The drawing systems currently in use generally provide for the use of test tubes having a pre-existing vacuum therein and the use of further components which allow the transfer of the blood from the patient to the test tube.

In particular, these systems use components provided with a further sharpened end, in addition to that used for drawing, covered with a layer of latex and which is also mounted on a device commonly known as holder. The transfer of blood inside the test tube occurs by inserting the needle, used for the drawing, in the vein of the patient and inserting the test tube inside the holder, provided with the aforesaid sharpened end covered in latex. The holder is also connected to the needle, usually a butterfly needle, by means of a small flexible connection tube.

The test tubes used for sampling blood are generally provided with a cap provided with a membrane made of perforable rubber. This characteristic ensures that the insertion of one such type of test tube, inside the holder, determines the perforation of the rubber membrane, by the sharpened end of the holder, and the consequent passage of blood from the patient to the test tube, the latter also having a predetermined internal vacuum.

As stated above, in some cases it is necessary to use common syringes, for example when it is necessary to graduate the suction, rather than the devices described above. Moreover, the latter—involving the use of components such as holders, connectors, butterfly needles and primary test tubes necessarily having a pre-existing vacuum at their interior—in specific circumstances have a relatively high cost.

For such purpose, the present invention proposes a new system for sampling biological material and in particular for sampling venous blood. In particular, the present invention, which will be described in detail hereinbelow, proposes a kit of components adapted to make the transfer of the drawn biological material into test tubes used for sampling easy, fast and safe. In addition, the present kit, given that it is to be used in situations requiring the use of common syringes as well as common collection test tubes, such as those having an inert atmosphere therein rather than a predefined vacuum, has a decidedly lower cost than those of current systems for blood drawing, comprising holders, connectors, butterfly needles and vacuum test tubes.

The safety conditions reducing the risk of contamination from biological material—ensured by the kit—and its relatively low manufacturing cost render the object of the invention particularly suited to be used in contexts and in places where adequate prophylaxis measures have not yet been set or where health practices that may not conform with hygienic norms are still in operation, as often occurs in less developed countries. In the latter, many health procedures, like vaccinations, are not practiced or are incomplete and the hygienic-health conditions are decidedly precarious.

### DESCRIPTION OF THE INVENTION

The present invention describes a new kit to be used during the drawing of biological material and in particular during the drawing of blood. More in detail, the invention describes a kit to be used during the step pertaining to the transfer of venous blood from a common syringe to a common collection test tube used for sampling the previously drawn substance. More in detail, the present kit, in its simplest embodiment, comprises at least one casing to be assembled, or already assembled, to a further component which is characterized in that it has at least two projections of which one is represented by a sharpened end, and more in detail by a needle preferably covered in latex, and another is represented by a conical portion adaptable to the neck of a common syringe. Said projections are also oriented in opposite direction with respect to each other and delimited by a disc-shaped base that is preferably integral with said projections or is integral with the casing. The joining of the aforesaid casing with the component having the above-described projections determines the obtainment of a connector adapted to facilitate the connection of the syringe, used for drawing the venous blood, with the test tube used for sampling the drawn fluid.

The kit, object of the invention, ensures that the step pertaining to the transfer of blood into the test tube occurs in conditions that reduce the risk of biological contamination and is also easy to execute, due to the particular structure of its components. More in detail, the casing with cylindrical shape of the kit according to the present industrial invention patent application, has, at the projections delimited by the disc-shaped base, open surfaces which facilitate the access of the syringe, on one side, and that of the collection test tube, on the other side. More in detail the operator, once he has performed the drawing of the biological material, and in particular of the venous blood from the patient, will have to insert the syringe, and in particular its neck, from which the drawing needle has been previously removed, inside the casing portion having the projection adaptable to the neck of the syringe, and then insert the test tube inside the opposite casing portion provided with the needle covered in latex. The latter, as stated above, is used for perforating the membrane usually present on the cap of the common test tubes. The casing of the kit, as will be described below in a more detailed manner, is shaped and sized in a manner such to reduce, if not eliminate, the risk of infections due to accidental punctures or, in any case, due to contact with the possibly-infected drawn blood. As mentioned above, the kit described in the present document comprises a protection casing that can either be assembled to the component having the projections, possibly integral with the disc-shaped base, or that is already firmly joined to said component. As will be described hereinbelow, the present kit, in some embodiments, comprises at least one sterile package, represented by a plastic bag or by a case and adapted to contain the components of the kit, i.e. to contain the casing(s) and the components to be assembled thereto.

In particular, in some embodiments the package comprises a plurality of components with double projection and an equal number of casings to be assembled or already assembled to said components. In addition, the package of the kit may contain a plurality of components with double projection and a single casing to be reused, the latter, for the various samplings, in any case ensuring that adequate safety conditions are present for reducing the risk of contamination from biological material.

### BRIEF DESCRIPTION OF THE DRAWINGS

FIG. 1 shows a view of the kit 1 according to the present invention and the components present therein. More in detail, FIG. 1(a) shows a side view of the protection casing 2, comprised in the kit 1, and the component 4 present inside the protection casing 2. FIG. 1(a) underlines the fact that said protection casing 2 has at least two portions, and in particular a portion 2′ and a portion 2″. The protection casing 2 also has cylindrical form and has its circular bases open. In particular the portion 2′ has the circular base 3′ open, and the portion 2″ has the circular base 3″ open, in a manner such to allow the entry of a common syringe into one, a common test tube into the other. As is observed in the figure, also the component 4 substantially has at least two main portions, and in particular a to projection 4′, with conical-shaped and internally hollow and adapted to constitute the connection element with the front part of the syringe 30 (the latter not shown in the figure), and a projection 4″ represented by a needle possibly covered in latex and adapted to allow the connection with the test tube 40 (not shown in the figure) and more in detail to facilitate the perforation of the rubber membrane usually present on the cap of the common test tubes for the sampling of blood.

FIG. 1(a) also shows that the projection 4′ is only partly contained inside the portion 2′ and, in particular, has the circular end thereof, i.e. the base of the cone, adapted to be screwed and/or fit in the neck of the syringe, extended outside said portion 2′. FIG. 1(b) is similar to FIG. 1(a), but, as is observable, the projection 4′ is in this case completely contained inside the portion 2′ of the protection casing 2. FIG. 1(c) shows a front view of the package adapted to contain the elements of the kit 1 according to the present invention. As is observed in the figure, the package 1 is represented by a common case or, more simply, by a common plastic bag. In the case in which the package 15 is represented by a case, the latter has preferably a plurality of housings 16 adapted to arrange, in an orderly manner, the elements of the kit 1 inside the package 15.

FIG. 2 shows a view of the protection casing 2 and the component 4 of the kit 1 in a particular embodiment. More in detail FIG. 2(a) shows a front and side view of the protection casing 2, in which its portion 2′ and its portion 2″, of equal extension, are more clearly observable. The two portions, 2′ and 2″, are also separated by a disc-shaped base 5 which is, in this case, integral with the same protection casing 2 and is extended along the central cross section of the protection casing 2. As is observed in the figure, the disc-shaped base 5 centrally has a hole 6 adapted for the traversing of the projection 4″ of the component 4, and a housing 19, extended into the portion 2′ adapted for the reversible fixing, by means of insertion, screwing and/or fitting, of the component 4. The component 4 is indeed assembled to the protection casing 2 by inserting the projection 4″ in the housing 19 and fixing, by means of screwing and/or fitting, the body of said component 4 inside the housing 19. Said component 4, as is observed in the figure, in addition to having the two opposite projections 4′ and 4″, has a welding element 20 adapted to firmly and irreversibly connect the two projections 4′ and 4″ to each other, said welding element 19 has cylindrical shape and has, at the zone of junction with the projection 4″, a thread 21 or a common fitting system, adapted to fix the component 4 with the housing 19 of the protection casing 2. The latter can also optionally comprise tabs 18 extended orthogonally with respect to the external surface of said protection casing 2. FIG. 2(b) shows the case in which the component 4 is assembled, as described in FIG. 2(a), to the protection casing 2.

FIG. 3 shows a view of the protection casing 2 and the component 4 assemblable thereto, in a particular embodiment of the kit 1 according to the present invention. More in detail, FIG. 3(a) shows the particular case in which the protection casing 2 is openable and closeable longitudinally in a manner so as to facilitate the insertion and housing of the component 4 therein which, in this case, has the disc-shaped base 5 firmly assembled thereto. The figure shows that the protection casing 2 is openable at the opening 9 directed longitudinally along the entire extension of the casing. The protection casing 2 is therefore openable and reclosable into two longitudinal portions 7′ and 7″, whereas the component 4 has the disc-shaped base 5 between the cylindrical welding 20 and the thread 21. FIG. 3(b) shows a perspective view of the protection casing 2 described in FIG. 3(a). More in detail FIG. 3(b) shows the protection casing 2 in open configuration, i.e. in the configuration adapted to receive the assemblable component 4.

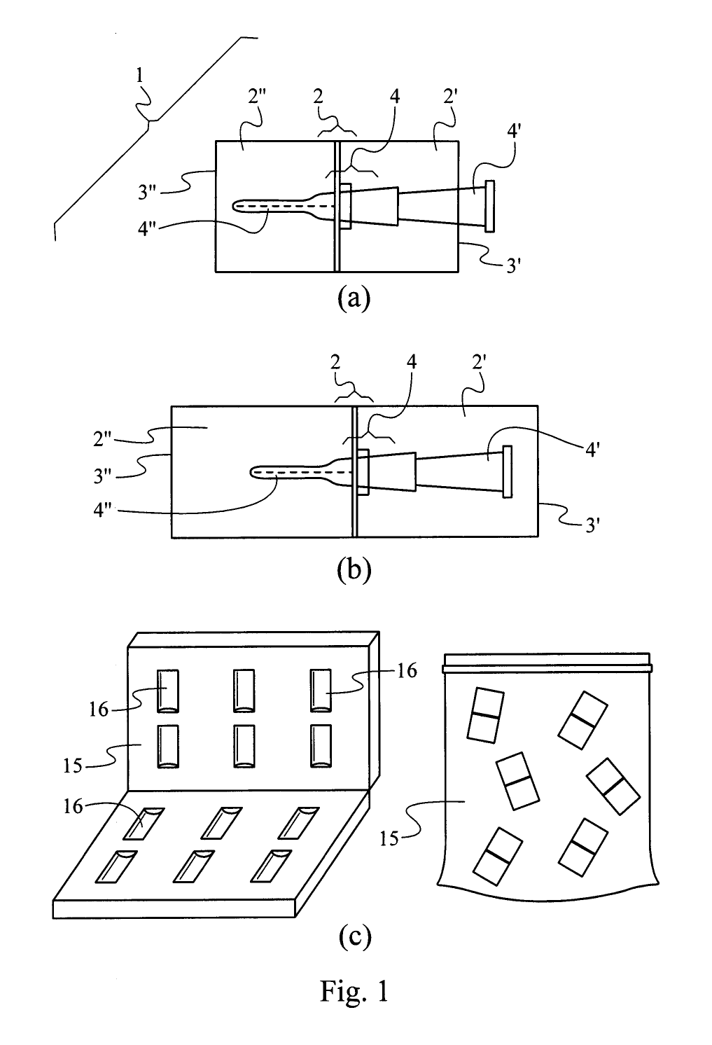

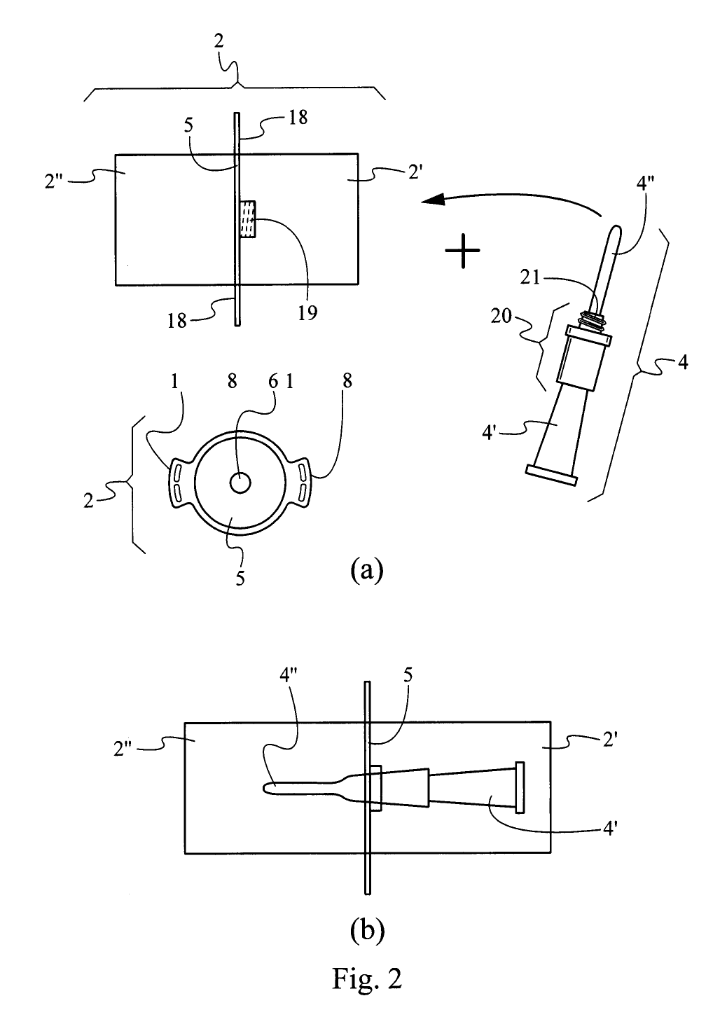

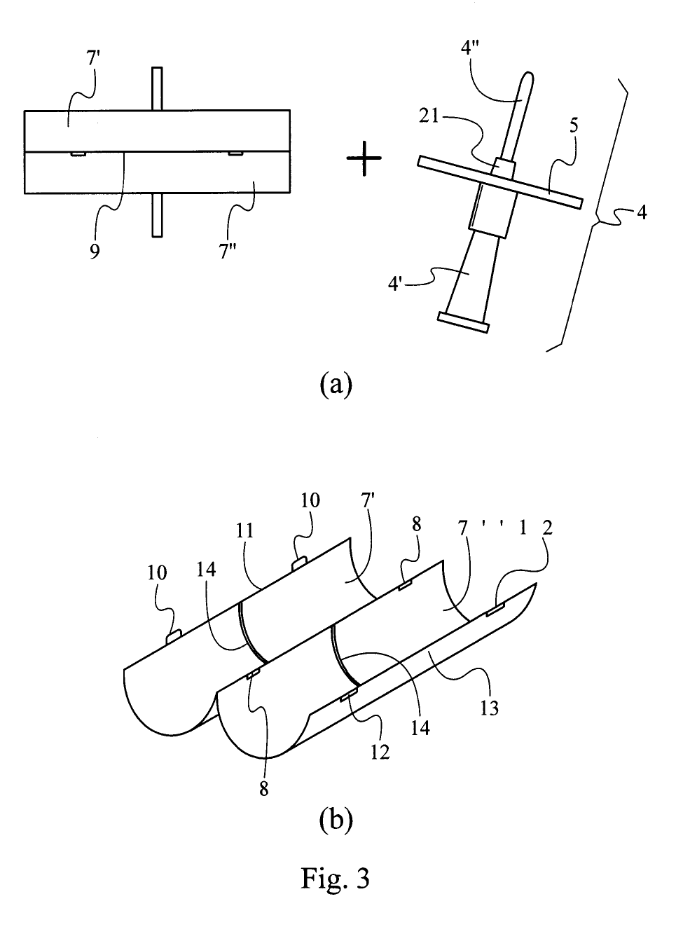

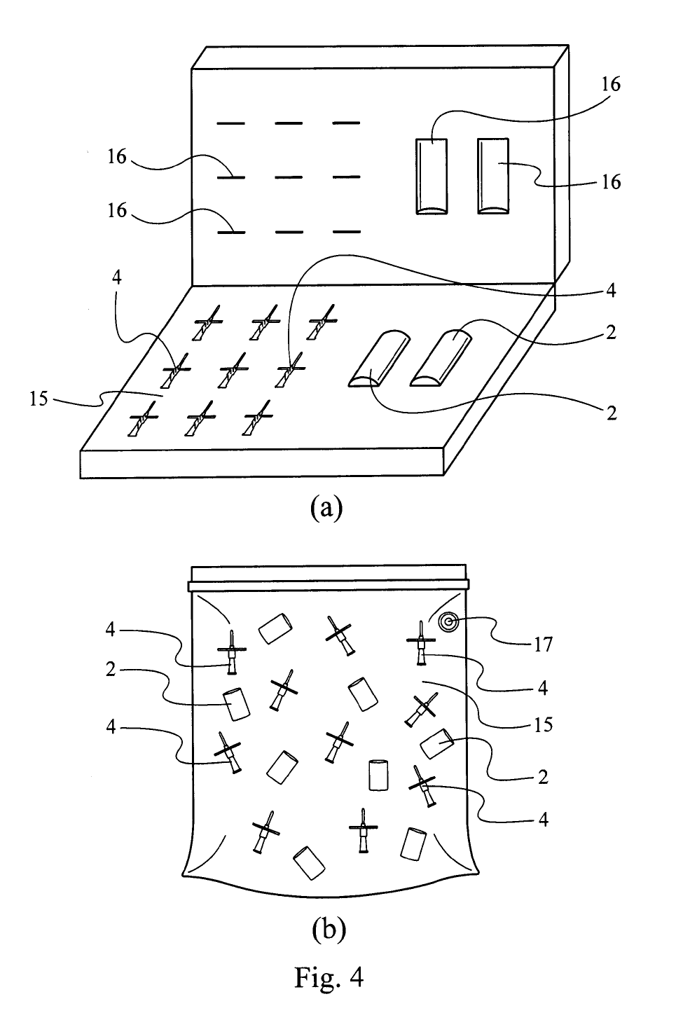

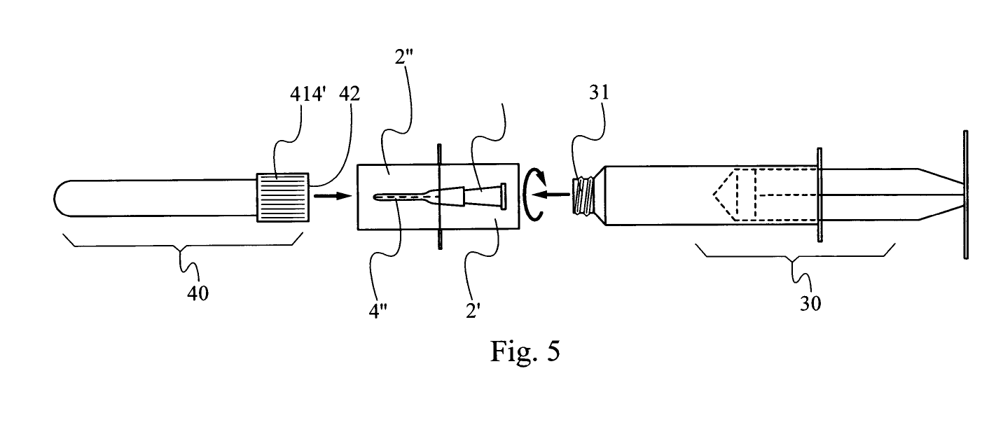

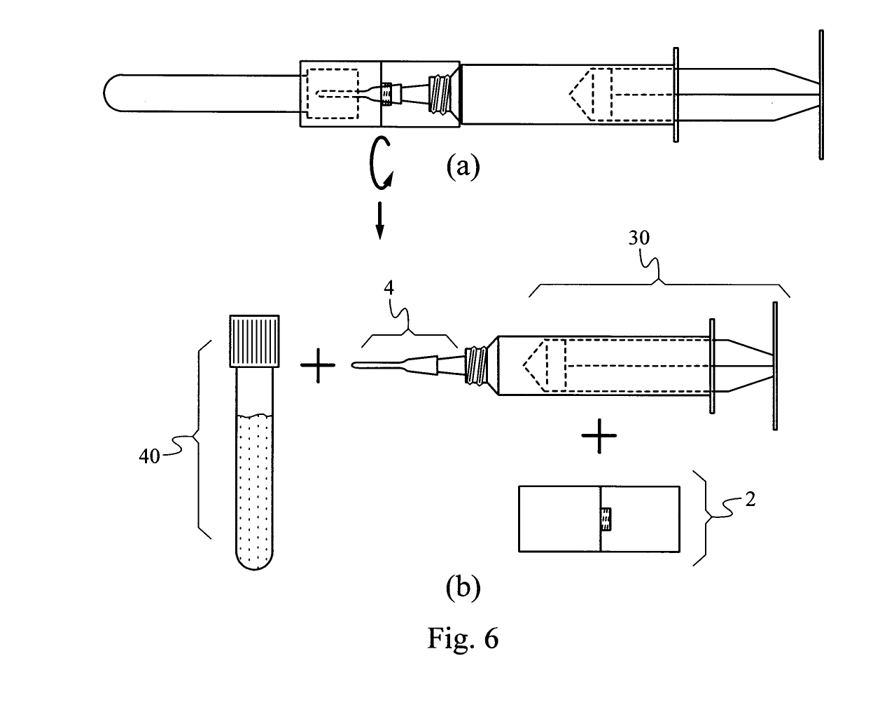

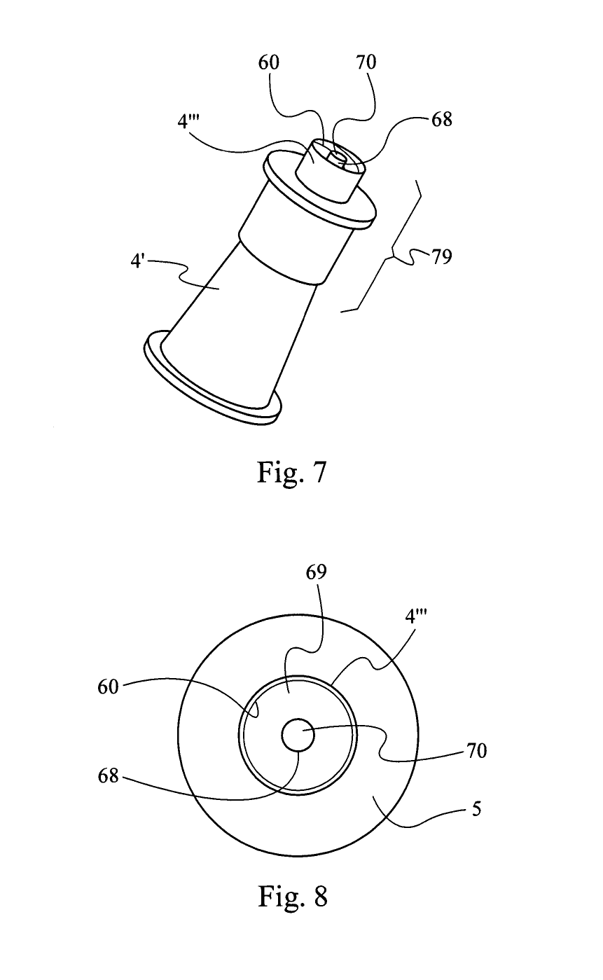

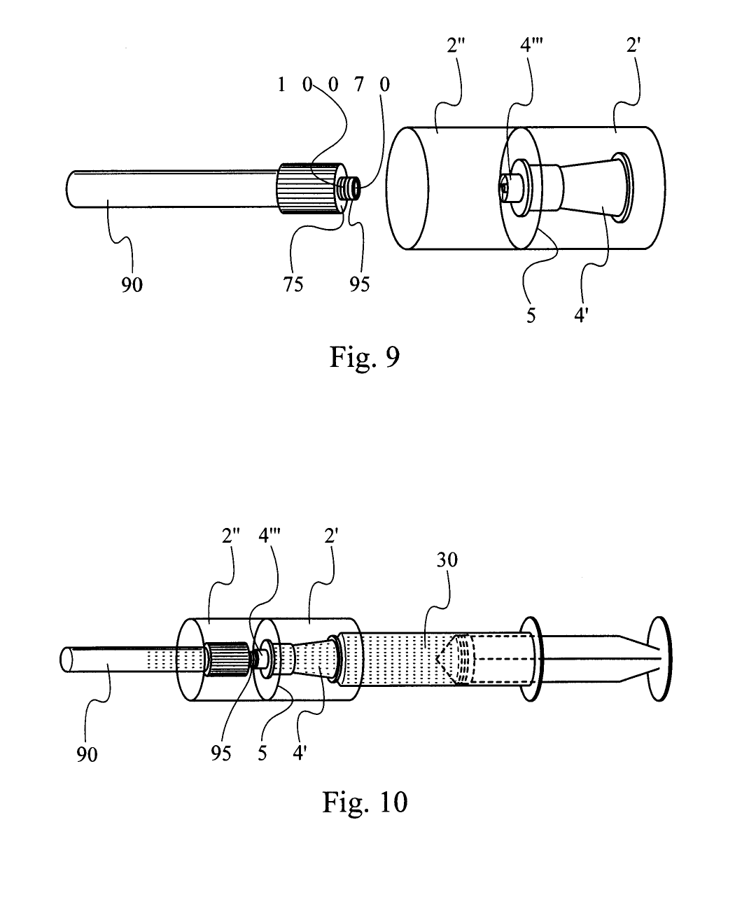

It is observed in the figure that the two longitudinal portions are joined due to at least one welding 8, present at the generatrix of the cylinder, representing the protection casing 2, opposite that indicating the opening 9. The portion 7′ also has, at its linear edge 11, at least one projection 10 adapted to be reversibly inserted and locked, with mechanisms of known type, inside a corresponding slot(s) 12 placed on the linear edge 13 of the corresponding longitudinal portion 7″. FIG. 3(b) also shows that, inside the protection casing 2, a groove 14 is present, transverse and central with respect to said protection casing 2 and adapted to constitute the housing for the disc-shaped base 5 of the component 4.

FIG. 4 shows a view of the package 15 of the kit 1 according to the present invention. More in detail FIG. 4(a) shows a perspective view of the package 15, when this is represented by a common case. The figure shows that the package contains, in this case, a plurality of housings 16 adapted for the ordered arrangement of the assemblable components 4 and of the protection casing 2. The latter can be present in a number equal to that of the components 4 or in a decidedly smaller number, given that the protection casing 2 is possibly reusable, while the components 4 are disposable. FIG. 4(b) instead shows the case in which the package 15 is represented by a common plastic bag that is sealed with expedients of known type. FIG. 4(b) shows that the package 15 contains, in this case, a plurality of components 4 and casings 2 distributed inside the bag in a random manner. The figure also shows that the bag representing the package 15 optionally comprises at least one valve 17 adapted to remove the air from the bag in a manner so as to preserve the elements comprised in the kit 1 under vacuum.

FIG. 5 shows a side view of the mode of use of the components of the kit 1 according to the present invention. In particular, FIG. 5 shows a side view relative to the insertion of a common syringe 30, and more in detail of its neck 31 inside the portion 2′ of the protection casing 2. The figure also shows the presence of the test tube 40 adapted to be connected with the portion 2″ of the protection casing 2. More in detail the figure shows that the insertion of the cap 41 of the test tube 40 inside the portion 2″ will cause the perforation of the rubber membrane 42 present on the cap of the test tube. The perforation will occur due to the needle possibly covered in latex representing the projection 4″ of the component 4. The test tube 40 can be provided with interior under vacuum or it can be a common sterile test tube.

FIG. 6 shows a view of the steps subsequent to the joining of the syringe 30 and the test tube 40 to the components of the kit 1 according to the present invention. More in detail FIG. 6(a) shows the step of transferring the biological material, previously drawn, from the syringe 30 to the test tube 40 and that this can occur by manually acting on the plunger of the syringe or by automatically exploiting the vacuum present in the test tube 50. Hence, as is observable in the figure, all the components involved in the transfer step are suitable assembled together here. FIG. 6(b) shows the step immediately following that pertaining to the transfer of the substance drawn inside the test tube 40. More in detail FIG. 6(b) shows the test tube 40 filled with the substance under examination, the protection casing 2 disengaged from the test tube, from the syringe and from the component 4, therefore resulting possibly reusable, and the syringe 30 which will be eliminated, together with the component 4 connected thereto, constituting of course a disposable object. As can be deduced from the figure, all the steps of interest for the invention are carried out under safe conditions; this reduces, if not fully eliminates, the risk of contamination from biological material. The thread of the neck 31 of the syringe 30 will have a screwing direction on the external face of the component 4′ analogous to the screwing direction of the thread 21 placed on the element 20.

FIGS. 7 and 8 show the device 20 in which in place of the projecting element 4″, there is the device 79. The device 79 is constituted by two concentric cylinders, the more external cylinder 4′ and the more internal cylinder 68, joined together by means of their lower edge or floor 69. The central cylinder 68 has the hole 70 placed centrally therein, adapted to allow the passage of the fluids or blood. The external cylinder 4″′ has instead, on the inner face thereof, the thread 60 adapted to be engaged on the corresponding thread 100 placed on the external face of the cylinder 95 arranged at the end of the test tube 90.

FIGS. 9 and 10 show the cylinder 4″′ placed inside the protection casing 2, adapted to be engaged, due to the thread 60, with the corresponding external thread 100 placed on the external face of the cylinder 95. The screwing of the cylinder 95 inside the cylinder 4″′ due to the respective threads 100 and 60 allows the progressive approaching of the central cylinder 68 to the hermetic polymer membrane placed at the base of the cylinder 95, and then, by continuing to screw the two elements, to the breakthrough of such membrane, placing in direct contact the liquid contained in the syringe 30 with the test tube 90 due to the presence of the holes 70.

### DESCRIPTION OF THE EMBODIMENTS

In a first embodiment, the kit 1 for treating biological material, and in particular for the transfer of venous blood from a common syringe to a common test tube, comprises: at least one protection casing 2, with cylindrical tubular form in which two transverse portions with equal size are identified, i.e. the portion 2′ and the portion 2″. The latter also have their circular bases open. More in detail the portion 2′ has the base 3′ open and the portion 2″ has the base 3″ open. Given that the protection casing 2 is cylindrical and internally hollow, it allows the insertion and assembly of a further component of the kit 1 according to the present invention. More in detail, the protection casing 2 is shaped and sized in a manner such to receive at least one component 4 with double projection, adapted to indirectly connect a common syringe 30 for drawing biological material, and preferably for drawing venous blood, with a common collection test tube 40.

Still more in detail, the component 4 of the kit 1 according to the present invention, is characterized in that it has at least one conical-shaped projection 4′, internally hollow, and adapted to constitute the element of connection with the syringe 30, and at least one projection 4″, represented by a common needle possibly covered by a latex covering, and adapted for the connection with the test tube 40. The two projections 4′ and 4″ are also firmly connected to each other by means of a cylindrical welding element 20 having a thread 21 at the zone of junction with the projection 4″. In addition the projection 4′ is extended into the portion 2′ of the protection casing 2, while the projection 4″, opposite the projection 4′, is extended into the portion 2″. The projection 4′, moreover, can be found entirely within the portion 2′, or only partly within such portion, i.e. having the circular end of its conical portion exiting from said portion 2′. Said component 4, as well as its projections 4′ and 4″ to are as stated above adapted to indirectly connect a common syringe 30 with a common collection test tube 40. In particular the projection 4′ is to be assembled by screwing and/or fitting to the neck 31 of the syringe 30, while the projection 4″ is adapted to perforate the membrane 42 present on the cap 41 of the test tube 40. The perforation occurs by manually inserting the test tube 40 and in particular its cap 41 inside the portion 2″ of the protection casing 2. A further element that delimits the two projections 4′ and 4″ from each other is represented by a disc-shaped base 5 which is, in some embodiments, integral with the same protection casing 2 and which also separates the latter into its two separate portions 2′ and 2″.

The disc-shaped base 5 has centrally a hole 6 adapted for the insertion and locking of the component 4. In particular, in the first embodiment the components of the kit 1 according to the present document, and in particular the protection casing 2 and the component 4, are such to be firmly and irreversibly assembled to each other. In other words, the aforesaid components of the kit 1 constitute a single element. In another embodiment, the component 4 is instead to be assembled to the protection casing 2 via insertion and reversible locking of said component 4 in the hole 6 of the disc-shaped base 5, in this case integral with the protection casing 2. The disc-shaped base 5, in addition to the hole 6, has a cylindrical housing 19 extended into the portion 2′ of the protection casing 2 and situated at the same hole 6 present on the disc-shaped base 5. All the components constituting the invention, including the syringe 30 and the test tube 40, are all indeed internally hollow in a manner so as to allow the passage of the drawn substance, generally blood, from one component to the other, and in particular from the syringe 30 to the test tube 40.

Inside said housing 19, the projection 4″ is then inserted, which by traversing the hole 6 is then extended into the portion 2″. The fixing of the component 4 occurs by screwing and/or fitting the thread 21 inside the housing 19 which will also have a corresponding thread on its internal surface.

In the case in which the component 4 is reversibly assembled to the protection casing 2, for example by fitting or by screwing, the operator can reuse the protection casing 2. In to particular, after having transferred the drawn blood by means of injection inside the test tube 40, and replaced the latter, the operator will have to remove the component 4, as well as the syringe 30, from the protection casing 2. The syringe 30 is indeed connected to the component 4 by screwing and/or fitting of the projection 4″, inserted and/or screwed, into the neck 31 of the syringe 30. Hence, with a simple rotary movement, opposite that carried out for the assembly, the operator will remove both the component 4 and the syringe 30, and will be able, if necessary, to reuse the protection casing 2. Otherwise, if said component 4 is already firmly and irreversibly connected to the protection casing 2, as described in the first embodiment of the present invention, the element resulting from the combination of the aforesaid components intuitively results disposable.

In a third embodiment, the kit 1 according to the described invention is characterized for the particular configuration of its components and in particular for the configuration of the protection casing 2 and the projection 4. More in detail, in this embodiment the component 4 is such to have the disc-shaped base 5 assembled therewith, so that the component 4 and the disc-shaped base 5 constitute a single element, to be subsequently assembled to the protection casing 2. The latter will therefore be configured in a manner such to have two longitudinal portions 7′ and 7″ that allow the opening of the protection casing 2 like a casket. More in detail the protection casing 2 is openable longitudinally at any one generatrix of the cylinder that represents said protection casing 2. The longitudinal portions 7′ and 7″ are moreover firmly connected to each other by means of at least one welding point 8, situated at the generatrix of the cylinder spatially opposite the generatrix indicating the opening 9 of the protection casing 2. The opening and the closing of the protection casing 2 is further facilitated due to the presence of at least one projection 10 placed on the linear edge 11 of the longitudinal portion 7′ and also due to the presence of at least one corresponding slot 12 placed along the linear edge 13 of the longitudinal portion 7″. Said projection(e) 10 is indeed adapted to be inserted and reversibly fit, with mechanisms of known type, inside the corresponding slot(s) 12.

In this embodiment, the protection casing 2 has a groove 14 at its interior and in the region that transversely separates said protection casing 2 into the portion 2′ and into the portion 2″. Such groove is adapted to facilitate the assembly of the component 4 to the protection casing 2. More in detail the groove 14 is shaped and sized in a manner such to allow constitute a housing for the disc-shaped base 5 which in this embodiment is integral with the component 4. In this case, the operator will then have to open the protection casing 2 and house the component 4 therein by positioning the disc-shaped base 5 in the groove 14, and finally reclosing the protection casing 2. The projection 4′ and the projection 4″ will consequently be oriented inside the protection casing 2 as in the above-described embodiments. Also in this case, the protection casing 2, after having transferred the drawn substance inside the test tube 40, can be reused, while the component 4 and the syringe 30 will be eliminated. The removal of the component 4 from the interior of the casing will occur by disengaging, with manual mechanisms of known type, the projection(s) 10 from the corresponding slot(s) 12 and manually removing the component 4.

In all its embodiments, the kit 1 according to the present invention further comprises at least one package 15 adapted to contain its components. In particular, in some embodiments the package 15 is represented by a common case having at least one protection casing 2 and at least one component 4 therein, the latter to be assembled or already assembled to said protection casing 2, as described in the present document in the first two embodiments. The package 15 is, more in detail, in some embodiments, a common plastic case, inside which the components of the kit are randomly arranged, or it is a common case in which the components are housed in an ordered manner therein, each component being placed in a suitable housing 16.

Alternatively, the package 15 of the kit 1 according to the present invention is represented by a common plastic bag, reversibly sealed with mechanisms and expedients of known type. The package 15, when represented by a common plastic bag, has in some embodiments a valve 17 adapted to remove the air from the bag, so that when said valve 17 is present, all the components of the kit are preserved under vacuum.

In all its embodiments, the package 15 is adapted to contain at least one protection casing 2 and at least one corresponding component 4. More in detail, in the case in which the package 15 is represented by a case, the latter will preferably contain six casings 2 to be assembled or already assembled to a corresponding number of components 4, or it will preferably contain at least one protection casing 2, possibly to be reused, and at least six disposable components 4 to be assembled to the protection casing(s) 2. In the case in which the package 15 is represented by a common plastic bag, the latter will contain at least one protection casing 2 and at least one component 4 assembled or assemblable as described in the various embodiments of the present kit 1, and preferably ten casings 2 and ten components 4 or at least one protection casing 2 and at least ten components 4.

In some embodiments of the kit 1 according to the present invention the package 15 is represented by a plastic bag containing over a hundred elements.

In a further embodiment of the present invention, the kit 1 can have, inside the portion 2″, the projection 79 (in place of the projection 4″) constituted by two concentric cylinders 4″′ and 68 respectively more external and more internal with respect to the central hole 70 and stably joined together by means of their internal portion i.e. the floor 69, given that said central cylinder 68 is provided with a hole 70 placed at the center thereof and adapted to allow the passage of the fluids or blood from the syringe 30 to the test tube 90. The external cylinder 4″′ has, on the inner face thereof, the thread 60 adapted to be engaged on the corresponding thread 100 placed on the external face of the cylinder 95 placed at the end of the test tube 90, in a manner such that the progressive screwing of the test tube 90 and especially of the cylinder thereof 95 into the external cylinder 4″′ first causes the approaching and then the breakthrough of the polymer membrane 75 present on the test tube 90, so as to place the contents of the syringe 30 in connection with the interior of the test tube 90. The central cylinder 68, due to the aforesaid screwing operation, first presses and then perforates the polymer membrane with which the end of the test tube 90 is equipped. Given that the zone broken by the central cylinder 68 is rather narrow, by performing the operation in reverse, i.e. by unscrewing the cylinder 95 of the test tube 90 from the interior of the external cylinder 4″′, one once again obtains the closure of the broken zone of the polymer membrane.

Naturally, when the central cylinder 68 is inserted inside the test tube 90, it is provided to introduce the desired quantity of serum or blood into the test tube 90.

1. A safety kit for treating biological material and preferably for the treatment of venous blood, adapted to facilitate the transfer of blood under safe conditions from a common syringe to a common collection test tube, the latter having at its interior a predefined vacuum or sterile atmosphere, comprising: at least one tubular protection casing, preferably cylindrical, at least one component assemblable or already assembled inside said protection casing, said protection casing and said component, when assembled, being adapted to constitute the connection element between a common syringe and a common collection test tube, and at least one package adapted to contain the components of said kit, wherein said protection casing has at least two portions, i.e. at least one portion and at least one portion of equivalent extension and adapted to receive at their interior the component, said component having at least two projections, i.e. the projection extended through the portion and the projection extended into the portion, respectively adapted to connect with a common syringe and with a common collection test tube, said projection being vaguely conical-shaped, internally hollow, having the circular end thereof adaptable by screwing and/or fitting inside the neck of a common syringe, and said projection, opposite the projection, being constituted by a common needle possibly covered in latex, adapted to perforate the membrane present on the cap of the collection test tube, and the entry of said neck into the portion and that of said cap into the portion of the protection casing being possible, the latter having at said portions and respectively the base with open circular shape and the base with open circular shape, in a manner so as to connect the contents of the syringe with the interior of the test tube due to the continuity created by the hole.

2. The kit according to claim 1 wherein the projection and the projection of the component are firmly and irreversibly joined to each other, said component having at least one cylindrical welding element joining said projection to said projection.

3. The kit according to claim 1 wherein the protection casing and the component are disposable, said components, i.e. said protection casing and said component being irreversibly welded to each other, constituting a single body.

4. The kit according to claim 1 wherein the protection casing is possibly reusable and the component is disposable, said component being contaminated by the drawn biological material and being reversibly assembled to the protection casing, said protection casing having the disc-shaped base at its interior, integral with said protection casing and arranged centrally and transverse to the protection casing, and said disc-shaped base having at least one hole at the center and a corresponding cylindrical housing, the latter extended into the portion and adapted for the reversible fixing of the component, said component having the welding element with at least one thread at the zone of junction with the projection adapted to be inserted and screwed inside the housing, the latter having a corresponding threaded inner surface.

5. The kit according to claim 1 wherein the protection casing is reusable and openable like a casket into two longitudinal portions, and in that the component is disposable, said protection casing having two longitudinal portions i.e. the portion and the portion openable at the opening coinciding with any one of the generatrix lines of the cylinder representing said protection casing and said portion and said portion being joined together due to at least one welding placed at the generatrix of the cylinder spatially opposite that coinciding with the opening, and said component having the disc-shaped base irreversibly assembled thereto, said disc-shaped base being welded to the component between the welding element and the thread, and said disc-shaped base being adapted to be reversibly and stably housed inside the protection casing, the latter having at least one groove at its interior at the central cross section of the cylinder representing said protection casing.

6. The kit according to claim 1 wherein the protection casing is openable and closeable due to the presence of at least one projection placed on the linear edge of the portion of the protection casing and due to the presence of at least one corresponding slot placed on the linear edge of the longitudinal portion of said protection casing.

7. The kit according to claim 1 wherein the package is represented by a common case adapted to contain the components of said kit in a random manner.

8. The kit according to claim 1 wherein it has, inside the portion, the projection constituted by two concentric cylinders respectively more external and more internal with respect to the central hole and stably joined together by means of the lower side thereof i.e. the floor, said central cylinder being provided with a hole placed at the center thereof and adapted to contain the passage of the fluids or blood from the syringe to the test tube, the external cylinder has on the inner face thereof the thread adapted to be engaged on the corresponding thread placed on the external face of the cylinder placed at the end of the test tube, in a manner such that the progressive screwing of the test tube, and especially of the cylinder thereof, into the external cylinder first causes the screwing and then the breakthrough of the polymer membrane so as to join the contents of the syringe with the interior of the test tube.

9. The kit according to claim 1 wherein the package is represented by a common plastic bag adapted to contain the elements of the kit randomly distributed therein.

10. The kit according to claim 1 wherein the package can optionally be provided with a common valve adapted to remove the air from the interior of the package, said components of the kit being consequently stored under vacuum.
